(19) United States
(12) Patent Application Publication
(10) Pub. No.: US 2010/0112618 A1
(43) Pub. Date: May 6, 2010

(54) Measurement of the Activity of a Kynurenine-Converting Enzyme and/or a Kynurenic-Acid, Anthranilic-Acid and/or 3-Hydroxykynurenine-Producing Enzyme

(76) Inventor: Halina Baran, Wien (AT)

Correspondence Address: CHALKER FLORES, LLP, 2711 LBJ FRWY, Suite 1036, DALLAS, TX, 75234, US

(21) Appl. No.: 12/593,057

(22) PCT Filed: Sep. 27, 2007
(86) PCT No.: PCT/AT2007/000452

§ 371 Date: Sep. 25, 2009

(30) Foreign Application Priority Data
Mar. 27, 2007 (AT) GM 195/2007

(51) Int. Cl. C12Q 1/52

(52) U.S. Cl. 435/16

(57) ABSTRACT

The present invention relates to a method of measuring the activity of a kynurenine-converting enzyme and/or a kynurenic-acid-, anthranilic-acid- and/or 3-hydroxykynurenine-producing enzyme, the method comprising the step of measuring the activity in the presence of an interfering sample, preferably selected from a CSF (cerebrospinal fluid) or serum, and detecting the conversion of kynurenine and/or kynurenic acid and/or anthranilic acid and/or 3-hydroxykynurenine.

The present invention relates to the field of determation of biological marker compounds.

The enzyme kynurenine aminotransferase (hereinafter abbreviated KAT) catalyzes the biosynthesis of kynurenic acid (KYNA) from kynurenine. Several enzymes at the periphery are responsible for KYNA formation, and rat liver exhibits the highest KAT activities (1). Human CSF (cerebrospinal fluid) and the serum exhibit little or even non-detectable KAT activities (2). The change in the kynurenine metabolism has been documented in neuroimmunologic, neuroinflammatory and neurodegenerative processes, including schizophrenia and depression. In these diseases, new clinical markers associated with the kynurenine metabolism are of particular interest.

Therefore, the present invention provides for a method of measuring the activity of a kynurenine-converting enzyme (e.g., kynurenine aminotransferase, kynureninase, kynurenine hydroxylase), a kynurenic-acid-, anthranilic-acid- and/or 3-hydroxykynurenine-producing enzyme, the method comprising the step of measuring the activity in the presence of an interfering sample, preferably selected from a biological liquid sample or bodily-fluid sample, in particular a CSF (cerebrospinal fluid) and/or serum sample, and detecting the conversion of kynurenine and/or kynurenic acid and/or anthranilic acid and/or 3-hydroxykynurenine. In bodily fluids, such as CSF and/or serum, portions are included which are interfering with the kynurenine-conversion activity. This interfering effect is being reduced (or increased) in patients suffering from several diseases. A comparison of two effects produced by two different dosages of an interfering sample preferably selected from CSF and/or serum gives a relation R_{HB} or R_{BK} which is associated with the pathology/disease. Consequently, the inventive method can be used for diagnostic purposes, as described below. Preferably, the reduction of kynurenine and/or the formation of kynurenic acid, anthranilic acid and/or 3-hydroxykynurenine is detected.

Preferably, the enzyme is a kynurenine aminotransferase (KAT), preferably KAT I, KAT II or KAT III. It is of course also possible to use any isolated or synthesized transferase similar to KAT I, KAT II or KAT III.

Preferably, the activity is derived from a kynurenine-converting enzyme and/or a kynurenic-acid-producing enzyme of a tissue sample, preferably a liver-tissue sample, preferably a tissue homogenate, more preferred an isolated or synthesized liver-tissue sample. KAT is an endogenous enzyme which is present in many tissues and which can be used unpurified or little purified, as is the case with a tissue sample or a homogenate. Such a tissue sample is preferably derived from a mammal, preferably a rodent, e.g. a rat, or from a human.

In the most preferred embodiments, the interfering sample, preferably a CSF sample and/or serum, is derived from a mammal, preferably from a human. The interfering sample can also be derived from a healthy test individual and used as a standard reference, or derived from a test individual suffering from a disease in which little inhibition or activation of the kynurenine conversion is expected. It is likewise possible to use different amounts of the interfering sample, preferably CSF and/or serum, and to construct an interference curve as a function of the amount of the interfering sample or the enzyme. It is also possible to select two specific amounts of the interfering sample (or the enzyme), and to determine the relation of the disclosed different effects on the conversions, without drawing a complete curve. These relations (R_{HB} and R_{BK}) can be used for diagnosing a specific disease (for example, R_{HB} ranges between 1.5 and 3.5, and R_{BK} between 0 and 2.5).

The method preferably comprises the step of comparing the activity to the activity of the kynurenine-converting enzyme and/or the kynurenic-acid-producing enzyme, preferably derived from a tissue sample, in the absence of the interfering sample or by using different amounts of the interfering sample or the enzyme.

In a further aspect, the present invention provides for a method of diagnosing a pathology associated with the kynurenine or kynurenic-acid metabolism by using the above-described (in-vitro) method, wherein the pathology is indicated by an activity reduction of less than 80%, preferably less than 60%, particularly preferred less than 50%, more preferred less than 40%, particularly preferred less than 30%, most preferred less than 20%, compared to the activity without the interfering component (control). The relation of the effects of different amounts of interfering sample, preferably selected from CSF and/or serum, can be used for a diagnosis method.

In particular embodiments, the pathology is a neuroimmunologic, neuroinflammatory or neurodegenerative pathology, in particular schizophrenia, depression or multiple sclerosis (MS).

In particular embodiments, a serum sample is used as the interfering sample. Surprisingly, similar inhibitory properties of the CFS has turned out to be also possible in serum samples which are easier use. According to the present invention, by “serum” all serum-containing bodily fluids including blood (with cellular components) or blood plasma (with coagulation factors) are understood, with serum itself being most preferred.

According to another aspect, the present invention provides for a kit, comprising a biological sample that includes a kynurenine-converting enzyme and/or a kynurenic-acid-producing enzyme, preferably together with a tissue sample or a homogenate, in particular a liver homogenate, appropriate buffers and kynurenine, preferably L-kynurenine, and optionally also comprising pyridoxal-5′-phosphate.

The kit can be used with the inventive method. The enzymes can preferably also be present in the form of a synthesized liver or a homogenate having similarity with KAT I, KAT II or KAT III or aminotransferase(s) with similar properties.

In the kit, the enzyme preferably is a kynurenine aminotransferase (KAT), preferably KAT I, KAT II, or KAT III.

Moreover, the kit preferably comprises an oxoacid, preferably selected from pyruvate, 3-hydroxypyruvate, 2-oxoglutarate, 2-oxoisovalerate, 2-oxoadipate, phenylpyruvate, 2-oxobutyrate, glyoxalate, oxaloacetate, 2-oxo-gamma-methiolbutyrate, 2-oxo-n-valerate, 2-oxo-n-caproate, and 2-oxoisocaproate.

It is likewise preferred that the kit comprises a protein-denaturating agent, preferably in a microcentrifuge tube.

The kit preferably comprises kynurenic-acid, anthranilic-acid and/or 3-hydroxykynurenine standards for measurement comparisons.

The present invention is further illustrated by the following examples without being limited thereto.

### EXAMPLES

### Example 1

Measurement of KATs (KAT I and KAT II) activities in liver in the presence of CSF and serum shows significantly lowered KATs activities.

CSF and serum significantly reduced KYNA formation (KAT I activity) in rat-liver homogenate by 70% (30% of the control), and KAT II activity of liver homogenate was moderately influenced by human CSF or serum. Two different amounts of CSF or serum were applied as a composition of the mixture in the KAT reaction for the diagnostic, and a relation of both effects was established. Human CSF or serum from the control test individual and from an MS patient showed a different effect on liver KAT I activity, i.e. on formation of KYNA and other kynurenine metabolites, such as anthranilic acid and 3-hydroxykynurenine.

CSF or serum from MS patients showed significantly weaker capability of reducing KAT I activity (60% of the control) in the liver homogenate, i.e. showed significantly higher formation of KYNA compared to the effect of CSF or serum from control test individuals, wherein the inhibition of KAT I was 20 to 30% from the control (3). KAT II activity of rat liver was moderately influenced by human CSF or serum.

### Example 2

### KAT Assay

The KAT assay is generally known, and was performed according to the published work (Baran et al., 2004). (KAT activity measurement was also published in 1994; 2000; 2004). For diagnostic purpose, the reaction cocktail contained a mixture of rat-liver homogenate and CSF or serum. L-kynurenine, pyruvate, pyridoxal-5′-phosphate, and 150 mM 2-amino-2-methyl-1-propranol (AMPOL) buffer, pH 9.6, for KAT I, or 150 mM Tris-acetate buffer, pH 7.0, for KAT II, in a total volume of 0.2 ml. After incubation (for 16 hrs; the time is variable) at 37° C. (98.6° F.), the reaction was determined by addition of 10 μl of 50% TCA. Subsequently, 1 ml of 0.1 M HCl was added, and denatured protein was removed by 10 min at 14,000 rpm. (Eppendorf Microfuge). The condition of the substrate L-kynurenine, pyruvate and pyridoxal 5′-phosphate is also variable and can be used according to already published work (1, 2, 3).

The measurement of KAT activity and/or of kynurenine metabolites, i.e. the formation of KYNA, can be done with different methods:

1. Assay by spectrophotometer, as described by Baran et al., 1994 (5)
2. Assay by HPLC and anthranilic acid, as described by Baran et al., 1995 (8) and also (6, 7)
3. Assay by radioenzymatic method, as described by Kepplinger et al., 2005 (3)

Ad1) The newly formed KYNA was determined spectrophotometrically at 333 nm (Knox, 1953)

Ad2) Measurement of KYNA by HPLC was performed according to Shibata, 1988 (9) and Swartz, 1990 (10) with a modification described by Baran et al., 1996. The obtained supernatant is applied to a Dowex 50 W cation exchange column, and KYNA was eluted with 2 ml of distilled water as described by Turski et al., 1989 (11), eluated and determined by HPLC coupled with fluorescence detection (Shibata et al., 1988; Swartz et al., 1990). The HPLC system used for analysis of KYNA and anthranilic acid and/or 3-hydroxykynurenine consisted of the following: pump (Shimadzu, LC-6A), fluorescence detector (Shimadzu, RF-535) set at an excitation wavelength of 340 nm and an emission wavelength of 398 nm, and a Shimadzu C-R5A Chromatopac Integrator. The mobile phase (isocratic system) consisted of 50 mM sodium acetate, 250 mM zinc acetate, and 4% acetonitril, pH 6.2, and was pumped through a column of 10 cm×0.4 cm (HR-80, C-18, particle size 3 |o, M, InChrom, Austria) at a flow rate of 1.0 ml/min, run at room temperature (23° C., 73.4° F.). The retention time of anthranilic acid and KYNA was approximately 3.5 and 5 min, with a sensitivity of 250 fmol and 150 fmol per injection (signal−noise relation=5).

Ad3) Radioenzymatic method can be performed according to the method described by Baran at al., 2004 and Kepplinger et al., 2005.

### REFERENCES

1. Okuno E, Nishikawa T, M Nakamura, (1996) Kynurenine aminotransferases in the rat. Localization and Characterization. Recent Advances in Tryptophan Research, edited by Graziella Allegri Filipini et al., Plenum Press, New Your, 1996.
2. B. Kepplinger, H. Baran, A. Kainz, H. Ferraz-Leite, J. Newcombe and P. Kalina (2005) Age-related increase of kynurenic acid in human cerebrospinal fluid: Positive corratio with IgG and Sa-microglobulin changes. Neurosignals, 14(3), 126-135.
3. Kepplinger B, Baran H, Kainz A, Zeiner D, Wallner J (2006) Cerebrospinal Fluid of Multiple Sclerosis patients exert significantly weaker inhibition of Kynurenine Aminotransferase I activity in rat liver homogenate. Multiple Sclerosis 2006; 12:S1-S228, P496
4. H. Baran, B. Kepplinger, M. Draxler and H. Ferraz-Leite (2004) Kynurenic acid metabolism in rat, piglet and human tissues. In European Society for Clinical Neuropharmacology by ed L. Battistin, International Proceedings MEDIMOND S.r.1. E505R9004, 227-231.
5. H. Baran, E. Okuno, R. Kido and R. Schwarcz (1994) Purification and characterization of kynurenine aminotransferase I from human brain. J. Neurochem., 62, 730-738.
6. H. Baran, J. A. Hainfellner, B. Kepplinger. P. R. Mazal, H. Schmid and H. Budka (2000) Kynurenic acid metabolism in the brain of HIV-1 infected patients. J. Neural. Transm., 107, 1127-1138.
7. Baran H, Gramer M, Honack D and W. Loscher Systemic administration of kainate induces marked increase of endogenous kynurenic acid in various brain regions and plasma of rats. Eur J Pharmacol 1995; 286: 167-175.
8. Shibata K. Fluorimetric microdetermination of kynurenic acid, an endogenous blocker of neurotoxicity, by high performance liquid chromatography. J Chromat 1988; 430: 376-380.
9. Swartz K J, Matson W R, MacGarvey U, Ryan E A, Beal M F. Measurement of kynurenic acid in mammalian brain extracts and cerebro-spinal fluid by high-performance liquid chromatography with fluorometric and coulometric electrode array detection. Anal Biochem 1990; 185: 363-376.
10. Turski W A, Gramsbergen J B P, Traitler H, Schwarcz R. Rat brain slices produce and liberate kynurenic acid upon expose to L-kynurenine. J Neurochem 1989; 52: 1629-1636.

1. A method of measuring the activity of a kynurenine-converting enzyme and/or a kynurenic-acid-, anthranilic-acid- and/or 3-hydroxykynurenine-producing enzyme, the method comprising the step of measuring the activity in the presence of an interfering sample, preferably selected from a CSF (cerebrospinal fluid) or serum sample, and detecting the conversion of kynurenine and/or kynurenic acid and/or anthranilic acid and/or 3-hydroxykynurenine.

2. The method according to claim 1, characterized in that the enzyme is a kynurenine aminotransferase (KAT), preferably KAT I, KAT II or KAT III.

3. The method according to claim 1, characterized in that the activity is derived from a kynurenine-converting enzyme and/or a kynurenic-acid-producing enzyme of a tissue sample, preferably a liver-tissue sample, more preferred an isolated or synthesized liver-tissue sample.

4. The method according to claim 3, characterized in that the tissue sample is a tissue homogenate.

5. The method according to claim 3, characterized in that the tissue sample is derived from a mammal, preferably a rodent or a human.

6. The method according to any one of claim 1, characterized in that the interfering sample, preferably a CSF and/or serum sample, is derived from a mammal, preferably a human.

7. The method according to any one of claim 1, comprising the step of comparing the activity to the activity of the kynurenine-converting enzyme and/or the kynurenic-acid-producing enzyme, preferably derived from a tissue sample, as described in claim 3, in the absence of the interfering sample or by using a different amount of the interfering sample or the enzyme.

8. A method of diagnosing a pathology associated with the kynurenine or kynurenic-acid metabolism by using the (in-vitro) method according to claim 1, wherein the pathology is indicated by an activity reduction of less than 80%, preferably less than 60%, particularly preferred less than 50%, more preferred less than 40%, particularly preferred less than 30%, most preferred less than 20%, compared to the activity without the interfering component (control).

9. The method according to claim 8, characterized in that the pathology is a neuroimmunologic, neuroinflammatory or neurodegenerative pathology.

10. The method according to claim 8, characterized in that the pathology is schizophrenia, depression or multiple sclerosis (MS).

11. The method according to claim 1 for measuring the activity of a kynurenine-converting enzyme and/or a kynurenic-acid-producing enzyme, characterized in that the conversion of kynurenine and/or kynurenic acid is detected.

12. The method according to claim 1, characterized in that the interfering sample is a serum sample and/or CSF.

13. A kit, comprising a biological sample that includes a kynurenine-converting enzyme and/or a kynurenic-acid-producing enzyme, preferably together with a tissue sample or a homogenate, in particular a liver homogenate or synthesized liver, appropriate buffers and kynurenine, preferably L-kynurenine, and optionally also comprising pyridoxal-5′-phosphate.

14. The kit according to claim 13, characterized in that the enzyme is a kynurenine aminotransferase (KAT), preferably KAT I, KAT II or KAT III.

15. The kit according to claim 13, further comprising an oxoacid, preferably selected from pyruvate, 3-hydroxypyruvate, 2-oxoglutarate, 2-oxoisovalerate, 2-oxoadipate, phenylpyruvate, 2-oxobutyrate, glyoxalate, oxaloacetate, 2-oxogamma-methiolbutyrate, 2-oxo-n-valerate, 2-oxo-n-caproate, and 2-oxoisocaproate.

16. The kit according to claim 13, further comprising a protein-denaturating agent, preferably in a microcentrifuge tube.

17. The kit according to claim 13, further comprising kynurenic-acid, anthranilic-acid and/or 3-hydroxykynurenine standards.

18. Use of a kit according to claim 13 for a method according to claim 1.
